(19) United States
(12) Patent Application Publication
(10) Pub. No.: US 2011/0233427 A1
(43) Pub. Date: Sep. 29, 2011

(54) MAGNETITE-SILVER HETERODIMER NANOPARTICLES AND THEIR PREPARATION AND USE FOR TWO-PHOTON FLUORESCENCE

(75) Inventors: Jackie Y. Ying, Singapore (SG); Jiang Jiang, Singagapore (SG); Hongwei Gu, Singapore (SG); Huilin Shao, Singapore (SG)

(73) Assignee: Agency for Science, Technology and Research

(21) Appl. No.: 12/809,560

(22) PCT Filed: Dec. 18, 2008
(86) PCT No.: PCT/SG2008/000490

§ 371 Date: Jun. 18, 2010

Related U.S. Application Data
(60) Provisional application No. 61/006,123, filed on Dec. 19, 2007.

(51) Int. Cl. G01J 1/58; C07K 5/08; C09K 11/06; B32B 5/16; B82Y 5/00

(52) U.S. Cl. 250/459.1; 530/331; 428/402; 252/301.16; 977/773

(57) ABSTRACT

A heterodimer particle is provided which comprises a first component particle comprising magnetite and a second component particle comprising silver. The second component particle may have a structure and a particle size selected to generate two-photon fluorescence emission. The heterodimer particle may be irradiated with light of a wavelength selected to induce two-photon fluorescence emission, which is then detected. Tetramethylammonium hydroxide may be bonded to the surface of the first component and glutathione may be bonded to the surface of the second component. The heterodimer particle may be formed by preparing a magnetite particle and growing a silver particle on the magnetite particle, in the presence of 1,2-dodecanediol as a reducing agent.

### CROSS-REFERENCE TO RELATED APPLICATION

This application claims the benefit of U.S. provisional application No. 61/006,123, filed Dec. 19, 2007, the entire contents of which are incorporated herein by reference.

### FIELD OF THE INVENTION

The present invention relates to magnetite-silver heterodimer nanoparticles, methods of their preparation, and their use for two-photon fluorescence, and target imaging and manipulation.

### BACKGROUND OF THE INVENTION

Two-photon fluorescence (TPF) is useful for a variety of applications in diverse fields, such as for detecting or imaging medical or biological targets, including cells. TPF is an optical process wherein a fluorophore is excited by near simultaneous absorption of two photons (on the order of femto-second (fs) or shorter) and subsequently emits a single photon. The emission photon has an energy that is higher than the energy of each excitation photon. In TPF, the excitation light may be infrared light and the emission light may be visible light. Not all fluorescent materials are suitable for TPF applications as the TPF cross-sections are very low in some fluorescent materials. Some materials with specific structures or shapes have been shown to exhibit stronger TPF responses.

### SUMMARY OF THE INVENTION

It is thus desirable to identify or provide new and alternative materials and structures that are suitable for two-photon fluorescence (TPF) applications. It is also desirable to provide more efficient and convenient processes for preparing such materials.

It has been discovered that heterodimer nanoparticles formed of a magnetite (Fe₃O₄) component and a silver (Ag) component having selected structure and size are suitable for use in TPF applications.

It has also been discovered that heterodimer particles formed of a magnetite component and a silver component can be conveniently prepared according to the exemplary methods described herein. The product yield can be increased when the heterodimer particles are prepared by growing the silver component on a seed magnetite particle in the presence of 1,2-dodecanediol as a reducing agent. The reaction time for the above step may be relatively short, such as about 30 minutes, when the seed magnetite particles are prepared by heating iron oleate dissolved in octadecene.

Further, it has been discovered that the heterodimer particles may be conveniently made water-soluble when glutathione (GSH) is bonded to the surface of the silver component and tetramethylammonium hydroxide (TMAH) is bonded to the surface of the magnetite component.

Therefore, according to an aspect of the present invention, there is provided a method, in which a heterodimer, particle is irradiated with light of a wavelength selected to induce two-photon fluorescence emission in the particle. The heterodimer particle comprises a first component particle comprising magnetite and a second component particle comprising silver. The second component particle has a structure and particle size selected to generate two-photon fluorescence emission in response to the irradiation. Two-photon fluorescence emission from the heterodimer particle is detected. The particle size of the second component particle may be about 10 nm or higher, such as from about 10 to about 15 nm. The second component particle may comprise a silver crystal, and may be generally spherical. The heterodimer particle may be attached to, or inside, a target during irradiation and detection. The target may be imaged based on the detected fluorescence. The target may be a cell. The heterodimer particle may also comprise TMAH bonded to a surface of the first component particle, and GSH bonded to a surface of the second component particle. The irradiation light wavelength may be about 800 to about 950 nm. The heterodimer particle may be moved, such as being manipulated, with a magnetic force.

In accordance with another aspect of the present invention, there is provided a particle. The particle comprises a first component comprising magnetite and a second component comprising silver. The first and second components form a heterodimer nanoparticle. TMAH is bonded to a surface of the first component and GSH is bonded to a surface of the second component. The second component may be generally spherical. The second component may have a size of about 2 to about 15 nm, such as about 10 to about 15 nm. The first component may have a generally spherical or cubic shape.

In accordance with a further aspect of the present invention, there is provided a method, in which a magnetite particle is prepared and a silver particle is grown on the magnetite particle, in the presence of a reducing agent, to form a heterodimer nanoparticle comprising the magnetite particle as a first component and the silver particle as a second component. The reducing agent is 1,2-dodecanediol. The magnetite particle may be prepared by a process that includes heating iron oleate dissolved in 1-octadecene to a temperature of about 320° C. for, e.g., about 30 minutes. The iron oleate dissolved in 1-octadecene may be heated in the presence of oleic acid or sodium oleate. The silver particle may be grown on the magnetite particle by heating a precursor solution to a temperature from about 60 to about 110° C. for about 10 to about 60 minutes, where the precursor solution comprises magnetite particles, a silver source, 1,2-dodecanediol, and an organic solvent. The precursor solution may also comprise oleylamine. The organic solvent may be hexane or toluene. The silver source may be silver acetate.

In accordance with another aspect of the present invention, there is provided a method of preparing a water-soluble particle from a heterodimer particle comprising a magnetite component and a silver component. In this method, TMAH is bonded to a surface of the magnetite component, and GSH is bonded to a surface of the silver component. The heterodimer particle, GSH, and TMAH may be mixed in a solution, such as an aqueous solution, to form the water-soluble particle.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 is a schematic block diagram illustrating an optical system for two-photon fluorescence imaging of a target;

FIG. 2 is a schematic diagram illustrating the target of FIG. 1;

FIG. 3 is a schematic diagram of a heterodimer particle in the target of FIG. 2;

FIG. 4 is a schematic diagram illustrating a heterodimer particle with functionalized surfaces;

FIG. 5 is a schematic diagram illustrating a preparation route for preparing the heterodimer particle of FIG. 4;

FIGS. 6 and 7 are transmission electron microscopy (TEM) images of sample magnetite nanoparticles, with generally spherical or cubic shapes respectively;

FIG. 8 is a X-ray diffraction (XRD) spectrum of sample magnetite nanoparticles;

FIGS. 9, 10, 11, and 12 are TEM images of sample magnetite-Ag heterodimer nanoparticles of different shapes and sizes respectively;

FIG. 13 is a XRD spectrum of sample magnetite-Ag heterodimer nanoparticles;

FIG. 14 is a line graph showing ultraviolet-visible (UV-Vis) absorption spectra of sample magnetite nanoparticles and heterodimer nanoparticles;

FIG. 15 is a line graph showing the magnetic hysteresis loops of sample magnetite nanoparticles and heterodimer nanoparticles;

FIG. 16 is a line graph showing a portion of FIG. 15 at an expanded scale;

FIG. 17 is a TEM image of sample hydrophilic heterodimer nanoparticles;

FIG. 18 is a line graph showing UV-Vis absorption spectra of sample hydrophobic and hydrophilic heterodimer nanoparticles, measured before and after a ligand exchange;

FIG. 19 is a bright-field transmission image of cells;

FIG. 20 is a two-photon fluorescence (TPF) image of the cells of FIG. 19;

FIG. 21 is a superposition of the images of FIGS. 19 and 20;

FIG. 22 is a bright-field transmission image of the cells loaded with sample hydrophilic heterodimer nanoparticles;

FIG. 23 is a TPF image of the cells of FIG. 22;

FIG. 24 is a superposition of the images of FIGS. 22 and 23;

FIG. 25 is a bright-field transmission image of the cells loaded with sample hydrophilic heterodimer nanoparticles, at a different loading concentration;

FIG. 26 is a TPF image of the cells of FIG. 25;

FIG. 27 is a superposition of the images of FIGS. 25 and 26; and

FIGS. 28 to 33 are sequential bright-field images of cells labeled with sample hydrophilic heterodimer nanoparticles, and moved by a magnetic force.

### DETAILED DESCRIPTION

In an exemplary embodiment of the present invention, a magnetic and fluorescent heterodimer particle is used in a two-photon fluorescence (TPF) detection or imaging.

The heterodimer particle includes a first component particle formed of magnetite (Fe₃O₄) and a second component particle formed of silver (Ag). The first and second components form a heterodimer nanoparticle.

A heterodimer particle refers to a particle in which two component particles are attached to each other. The shapes and sizes of the component particles may vary and may be different from each other. For example, one or both of the component particles may be spherical. In one embodiment, the heterodimer particle may have a generally calabash shape, or a generally dumbbell shape. In another embodiment, the magnetite component may have a generally cubic shape.

For clarity, the two component particles of a heterodimer particle do not form a core-shell structure. However, a component particle in the heterodimer particle may itself have a core-shell structure.

TPF emission can result from electron excitation due to (near) simultaneous absorption of two excitation photons, which in combination provide the energy required for the electron excitation. As can be appreciated, the excitation energy for each two-photon emission comes from two separate photons. The intrinsic likelihood of such excitation is thus typically low in most materials, even in many fluorescent materials. Further, the likelihood of TPF in a particle can vary depending on the structure and shape of the particle. Thus, not all fluorescent particles are suitable for TPF detection or imaging.

In the present embodiment, the second (silver) component particle should have a structure and particle size selected to generate TPF. The silver component particle may have a crystal structure, but it is not necessary. It has been surprisingly discovered that when the second (silver) component particle has a particle size of about 10 nm or higher, the heterodimer particle can generate two-photon fluorescence that is suitable for practical TPF application such as TPF detection or imaging. In a specific embodiment, the second component particle may have a particle size of about 10 to about 15 nm. The first component particle may also have a particle size in the nanometer range such as about 10 to about 50 nm.

The term “particle size” as used herein refers to the average diameter of the particle when the particle has a generally spherical shape. As particles may have non-spherical shapes and different sizes, the particle size refers to the average size of the particles when used in reference to multiple particles. When a particle has an irregular non-spherical shape, its particle size refers to its effective diameter, which is the diameter of a spherical particle that has the same volume as the non-spherical particle. In cases where the particle has a generally geometrical shape, such as a cubic shape, the particle size may refer to a characteristic dimension for that geometrical shape. For example, a cubic shape may be characterized by the length of its side. Nanoparticles typically refer to particles having a particle size of about 1 to about 100 nm. Particle sizes and size distribution of nanoparticles can be measured using optical or electronic imaging techniques, such as transmission electron microscopy (TEM) or suitable light scattering (e.g. dynamic light scattering) techniques. Such techniques can be readily understood and applied by persons skilled in the art for a given application.

Without being limited to any particular theory, the fluorescence from Ag nanoparticles can be expected to originate from radiative recombination of sp-band electrons and d-band holes, which can be expected to be enhanced by 4 to 6 orders of magnitude due to surface plasmons of nanocrystals, or rough metal surfaces. It is expected that the surface plasmon strength of a nanoparticle increases as the particle size increases. The plasmon resonance frequency is also dependent on the particle size (likely weakly), particle shape, and particle aggregation. In general, particle elongation and aggregation can result in shifting of the surface plasmon resonance frequency to a smaller value (thus a longer wavelength). Due to the larger scattering cross-sections of aggregated particles, the electrical field at the particle surfaces is substantially enhanced, which can in turn lead to amplification of various types of optical (electromagnetic) phenomena, including Raman scattering and fluorescence, particularly TPF.

It is expected that when the frequency of excitation light matches the surface plasmon resonance frequency, fluorescence will be enhanced due to resonance enhancement effect.

The irradiation for inducing TPF may be effected with light of a wavelength selected to induce TPF emission in the particle. For example, when the single-photon absorption wavelength of the particles is about 450 nm, the irradiation may be effected with a laser light having a wavelength of about 900 nm, or in the range of about 850 to about 950 nm. In another embodiment, the irradiation light may have a wavelength in the range of about 800 to about 950 nm. A suitable wavelength of the excitation light for TPF may vary depending on the fluorescence frequency of the target material. The wavelength for TPF is generally twice the wavelength for single-photon fluorescence. In order to produce TPF emission sufficiently strong for detection or imaging, the excitation light should have sufficient intensity. In this regard, pulsed light may be used to provide high-intensity pulses, with a relatively low time-averaged intensity. Typically, the excitation light will be focused into a small focus region. Typically, due to the strong dependence of TPF on excitation intensity, virtually no TPF will occur outside the focus region. Thus, TPF can be conveniently induced at selected focus regions in the target.

The two-photon fluorescence emission from the particle may be detected using any suitable detection technique or device. For example, an image may be generated based on the detected emission.

Conveniently, the heterodimer particle may be attached to a detection or imaging target, such as a target cell, or placed inside the target. For example, the target may be a cell and the heterodimer particle may be placed inside the cell (taken up by the cell) or attached to the surface of the cell.

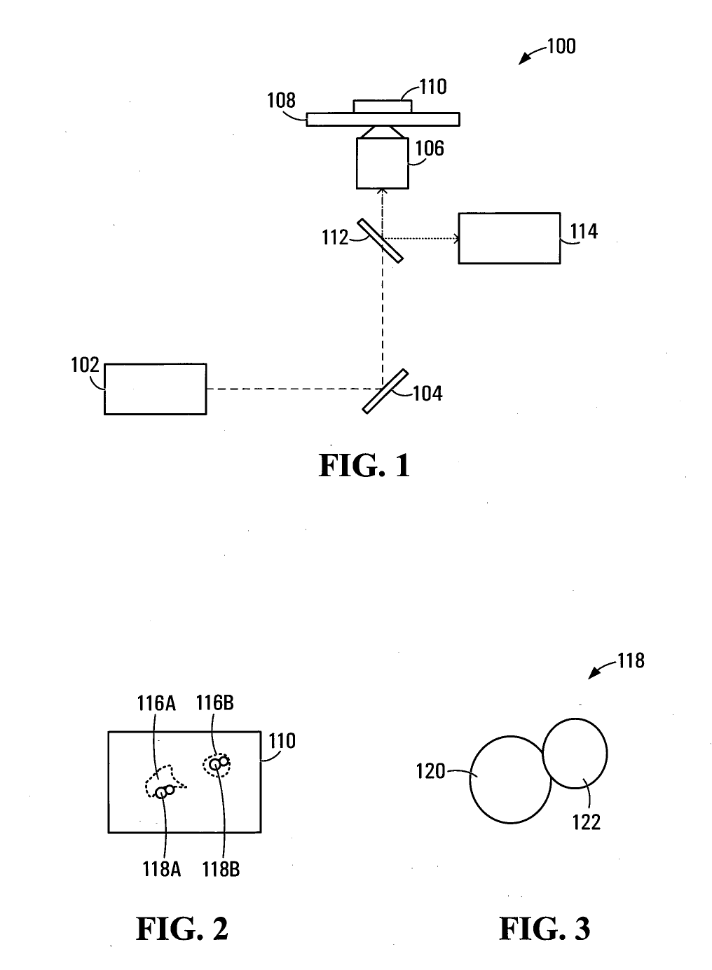

In an exemplary embodiment, TPF detection or imaging of cells may be performed using an optical system 100 illustrated in FIG. 1.

System 100 includes a laser source 102, which generates a beam of laser light (represented by the dashed line). The laser light may have a spectrum peak at a wavelength selected to induce TPF in the particular target material. For instance, the peak wavelength may be about 900 nm in some applications. The laser light may be directed by a scanner 104 towards a focusing device such as a microscope objective 106. Microscope objective 106 focuses the excitation light onto a target 110 supported by a target support 108. Target support 108 may be configured to adjust the position of target 110 with respect to microscope objective 106. Microscope objective 106 is selected to sufficiently focus the incident laser light onto target 110 so that TPF will be induced within the focus region.

TPF emission from target 110 (represented by the dotted-line in FIG. 1) is directed by a half-mirror 112 to a detector 114, which may be any suitable detector for detecting fluorescence emission or fluorescence imaging.

As schematically illustrated in FIG. 2, target 110 may include one or more cells, such as cells 116A and 116B, which are also individually and collectively referred to as cell 116, and one or more particles, such as particles 118A and 118B, which are also individually and collectively referred to as particles 118.

As schematically illustrated in FIG. 3, particle 118 is a heterodimer particle formed of a magnetite component particle 120 and a silver (Ag) component particle 122. Ag component particle 122 has a structure and size selected to generate and enhance TPF, and may have the exemplary structures and sizes described herein.

System 100 may also include other instruments and components (not shown) for, e.g., system control, signal conversion, data acquisition, data analysis, image processing, or the like.

In use, the excitation light is focused to a particular region in target 110 to induce TPF in that region. Scanner 104, microscope objective 106 and target support 108 may be adjusted to position the focus region at a selected point in target 110, as can be understood by those skilled in the art. The focused excitation light induces TPF in target 110, which is generated by particle 118 in response to the irradiation of the excitation light. The TPF emission is directed by half-mirror 112 to detector 114 and is detected by detector 114.

When detector 114 includes or is connected with an imaging component or device, an image of target 110 may be generated based on detected TPF signal using a suitable technique.

Before, during or after the detection or imaging process, particles 118 and cells 116 may be conveniently manipulated or moved with a magnetic force, such as using a magnetic field or magnet.

Particles 118 may be conveniently attached to, or taken up by, cells 116 by dispersing particles 118 in a cell culture medium (not shown) and contact cells 116 with the culture medium, such as culturing cells 116 in the culture medium.

Many techniques for attaching metal-based nanoparticles to cells have been described in the literature, including techniques for labeling and tagging cells for fluorescence detection, imaging or analysis. See, for example, I. L. Medintz et al., “Quantum Dot Bioconjugates for Imaging, Labeling and Sensing,” Nature Materials 4, 435 (2005), the entire contents of which are incorporated herein by reference. Some of these known techniques may be adapted or modified to attach particles 118 to cells 116, or to place particles 118 inside cells 116.

In one embodiment, particles 118 may be placed in contact with cells 116 so that cells 116 can take up particles 118 by cellular endocytosis.

Depending on the mechanisms used for attaching particles 118 to cells 116, particles 118 may be surface modified so that the modified surface has affinity to the target cells, and is chemically and biologically compatible with the cells. The particle surface may also be modified so that the particles will have specific affinity to a given target or target cell. When a nanoparticle is attached to the cell surface, the cell may eventually uptake the nanoparticle through cellular endocytosis.

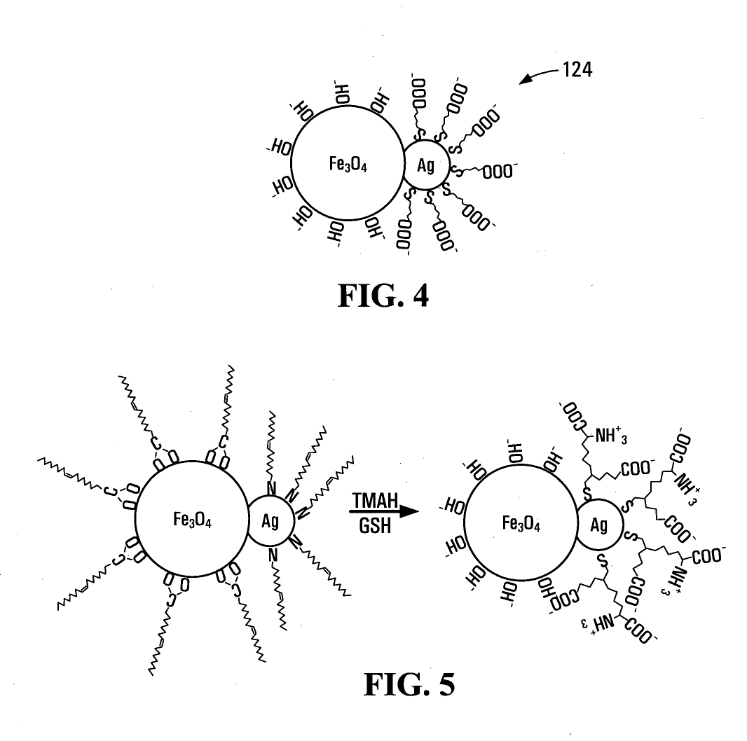

In some applications, it may be desirable that particles 118 are hydrophilic and soluble in water. In some embodiments of the present invention, the surfaces of the initially prepared heterodimer particles may be hydrophobic. For example, the surfaces of the component particles in the initial heterodimer particles may include a layer of oleic acid and oleylamine, both of which are hydrophobic. In such cases, the initial heterodimer particles may be functionalized with hydroxyl groups on the surface of the magnetite component, and with carboxyl and amine bearing thiol molecules on the surface of the Ag component, as schematically illustrated in FIG. 4. The heterodimer particle 124 illustrated in FIG. 4 is hydrophilic and is water-soluble. Thus, such particles can be conveniently dissolved in a cell culture medium or another aqueous solution and can be conveniently and be safely taken up or attached to cells 116.

Heterodimer particle 124 may be prepared according to an exemplary process described herein, by bonding glutathione (GSH) to the surface of the Ag component particle and bonding tetramethylammonium hydroxide (TMAH) to the surface of the magnetite component particle. In one embodiment, the surfaces of the initial heterodimer particles may be modified by mixing the initial (un-modified) heterodimer particles, GSH, and TMAH in an aqueous solution, to effect ligand exchange that displaces oleic acid and oleylamine with GSH and TMAH respectively. The corresponding ligand exchange process is schematically illustrated in FIG. 5.

In the above exemplary process for functionalizing the magnetite-Ag heterodimer particles, both the magnetite and the Ag particle surfaces can be modified at the same time, in a one-stage process. Thus, the above process can be advantageous over a conventional process in which different particle surfaces are modified at different stages, or in which pre-modification surface treatment is required.

As can be appreciated, particles like heterodimer particle 124 may have applications in other fields than TPF. Thus, another exemplary embodiment of the present invention relates to a water-soluble heterodimer particle. The particle is formed of a first component comprising magnetite, a second component comprising silver, TMAH bonded to magnetite on the surface of the first component; and GSH bonded to silver on the surface of the second component. The second component may be generally spherical, and may have a size of about 2 to about 15 nm, or about 10 to about 15 nm. As discussed above, when the second component has a size from about 10 to about 15 nm, the heterodimer particle may be conveniently used in two-photon fluorescence detection or imaging application. The first component may have a generally spherical or cubic shape.

When the water-soluble heterodimer particles are used in applications other than TPF applications, it may not be necessary to limit the particle size of the Ag component particle. A smaller size such as about 2 to about 10 nm may be suitable in some applications, such as in single-photon imaging or detection applications.

The heterodimer particles described herein may be prepared in different processes.

In an exemplary process, magnetite seed particles are initially prepared by heating iron oleate dissolved in 1-octadecene to a temperature of about 320° C. for about 30 minutes. The iron oleate dissolved in 1-octadecene may be heated in the presence of oleic acid or sodium oleate. The magnetite particles may be prepared using other techniques, such as by forming a mixture containing Fe-(acac)₃, phenyl ether, oleic acid, oleylamine and 1,2-hexadecanediol, and heating the mixture to 210° C. for about half an hour. However, it has been found that when the magnetite particles are prepared according to the former procedure, the time required for growing the silver component particles can be significantly reduced (such as by a factor of more than 8 as compared to the latter procedure).

Silver particles are grown on the prepared magnetite particles, in the presence of 1,2-dodecanediol as a reducing agent, to form the heterodimer particles. It has been discovered that while other reducing agents may also be used, a higher yield can be obtained when 1,2-dodecanediol is used as the reducing agent during silver particle growth. In one embodiment, a precursor solution containing magnetite particles, a silver source, 1,2-dodecanediol, and an organic solvent is heated to a temperature of about 60 to about 110° C. for about 10 to about 60 minutes. The precursor solution may also contain a surfactant, such as oleylamine. The organic solvent may be hexane or toluene. The silver source may be silver acetate, or another suitable silver precursor.

Conveniently, the shapes and sizes of the component particles may be independently controlled by adjusting the reagents (such as the amounts of precursor materials and the types of solvents or reducing agents), the reaction time, the reaction temperature, and other reaction conditions. How the shapes and sizes will change in response to these factors in a particular application can be determined by persons skilled in the art based on the known techniques disclosed in the literature or by conducting routine tests. Some examples and exemplary data for controlling particle shape and size with varying reaction conditions, and related literature references, are described in the Examples and Table I below.

Some variations of the above process may be possible if a particular feature or benefit discussed herein may be dispensed with to achieve a different objective. For example, other organic solvents may be used as the solvent. If a reduced yield is tolerable, another reducing agent may be used to replace 1,2-dodecanediol. If a reduction in the production rate during Ag-particle growth is tolerable, the initial magnetite particles may be prepared according to a different technique including other techniques known to persons skilled in the art.

While cells are used as the detection and imaging target in some examples described herein, the heterodimer particles described herein may be used with other targets and may even be itself the target of imaging or detection. For example, the heterodimer particles may be used in molecular imaging and nanoparticle self-assembly applications.

The variation and modification discussed above are for illustration purposes and are not exhaustive. Other variations and modifications to both the composites and their preparation processes are also possible.

While one may expect that the Ag component may be substituted by an Au component, test results have shown that a much higher yield can be obtained with a silver precursor in the preparation process for preparing heterodimer nanoparticles, as compared to Au.

The exemplary methods and processes described herein are also advantageous over some conventional techniques in that it is not necessary to pre-treat the precursor materials or particles, such as to perform initial surface modification, or to include additional stages of preparation which are required in such conventional techniques.

The exemplary particle preparation procedures described can be easy to perform, and can result in increased yield or higher production rate. The heterodimer particles may be prepared with selected sizes. The preparation conditions can be mild (e.g. at temperatures of less than about 110° C.). Thus, it is not necessary to use high temperature equipments or high boiling point organic solvents, which can add cost and complexity to the process.

As the heterodimer particles are magnetic, the heterodimer particles may be conveniently manipulated using a magnetic force and may be used in magnetic imaging applications. For example, as superparamagnetic iron oxide nanoparticles are expected to be good T2 contrast-enhancing agents, the heterodimer nanoparticles described herein may be conveniently used for magnetic resonance imaging (MRI) applications. Further, when the particles are attached to cells, the cells may be also be conveniently manipulated using a magnetic force or field. By targeting specific cell types, cell sorting and separation can be performed by application of a magnetic field.

Live cells labeled with heterodimer nanoparticles described herein have been successfully imaged using TPF microscopy, and manipulated using a permanent magnet, as further discussed in the Examples.

Embodiments of the present invention may be conveniently used in various applications, such as medical or biological applications. For example, they may be suitable for thick tissue and in vivo imaging or detection applications, in applications where multimodal imaging is desired, such as in cellular and sub-cellular TPF imaging, and in MRI imaging applications. They may also be useful for magnetic cell sorting and separation with real time optical monitoring.

The following examples are provided to assist understanding of the exemplary embodiments of the present invention.

### EXAMPLES

In the following examples, the following materials and instruments were used to perform the various tests and imaging described below.

Iron(III) chloride hexahydrate (Merck™, 99%), oleic acid (Aldrich™ tech. 90%), sodium oleate (TCI™, 95%), 1-octadecene (Aldrich, tech. 90%), oleylamine (Aldrich, tech. 70%), L-glutathione (GSH) reduced (Sigma-Aldrich™ 99%), tetramethylammonium hydroxide pentahydrate (Aldrich, 97%), 1,2-dodecanediol (Fluka™, 90%), and silver acetate (Lancaster™, 99%) were used as received without further purification.

An FEI Tecnai G² F20™ electron microscope operating at 200 kV was used for obtaining the TEM images. X-ray diffraction (XRD) patterns were obtained with a PANalytical X'Pert Pro™ Diffractometer. UV-Vis absorption spectra were taken with an Agilent™ 8453 UV-Visible Spectrophotometer. The atomic concentrations of the nanoparticle solutions were determined using ICP-MST™ (Elan DRC II Perkin-Elmer™). Two-photon fluorescence imaging was performed using a Zeiss™ LSM 510-Meta laser scanning microscope with a Ti:Sapphire laser (Mai Tai BB™, Spectra-Physics™) as the excitation source. Magnetization measurements were conducted using Quantum Design™ PPMS (Physical Properties Measurement System) and SQUID (Superconducting Quantum Interference Device) systems. The magnetization values of the samples were measured at 6K as a function of the applied field (up to 50 kOe).

### Example I

### Preparation of Magnetite Nanoparticles

Sample magnetite nanoparticles were synthesized by thermal decomposition of iron-oleate complex.

In a typical procedure, 2.7 g of FeCl₃-6H₂O 0 (10 mmol) and 9.125 g sodium oleate (30 mmol) were added to a mixture of ethanol (20 ml), deionized water (15 ml), and hexane (35 ml). The mixture was refluxed at 70° C. for 4 hours. An upper reddish brown hexane solution containing iron-oleate complex was then separated, and washed three times with deionized water (10 ml) in a separatory funnel. Hexane was then evaporated in a rotary evaporator, yielding a dark reddish brown, oily iron-oleate complex. Using a standard Schlenk line, iron oleate complex (9 g, 10 mmol) was dissolved in 25 g of 1-octadecene; oleic acid (1.41 g, 5 mmol) or sodium oleate (1.52 g, 5 mmol) was then added. The mixture was heated, under argon, to about 320° C. with a ramp rate of 3 to 5° C./min. The temperature was maintained at about 320° C. for about 30 minutes. The resulting black nanocrystal solution was cooled to room temperature, and 2-propanol (50 ml) was added to precipitate the magnetic nanoparticles. After centrifugation, nanoparticles were washed with hexane and ethanol three times, and then re-dispersed in hexane or toluene.

Magnetite nanoparticles with different sizes and shapes were prepared by changing experimental conditions, such as reaction temperature, and surfactant type and concentration. The preparation conditions were adjusted according to the procedures described in N. R. Jana et al., Chem. Mater., 2004, vol. 16, p. 3931; J. Park et al., Nat. Mater., 2004, vol. 3, p. 891; and M. V. Kovalenko et al., J. Am. Chem. Soc., 2007, vol. 129, p. 6352, the entire contents of each of which are incorporated herein by reference.

Both spherical and cubic shaped magnetite nanoparticles were prepared. Sample spherical particles were prepared by using oleic acid as the surfactant and cubic particles were prepared by using sodium oleate as the surfactant.

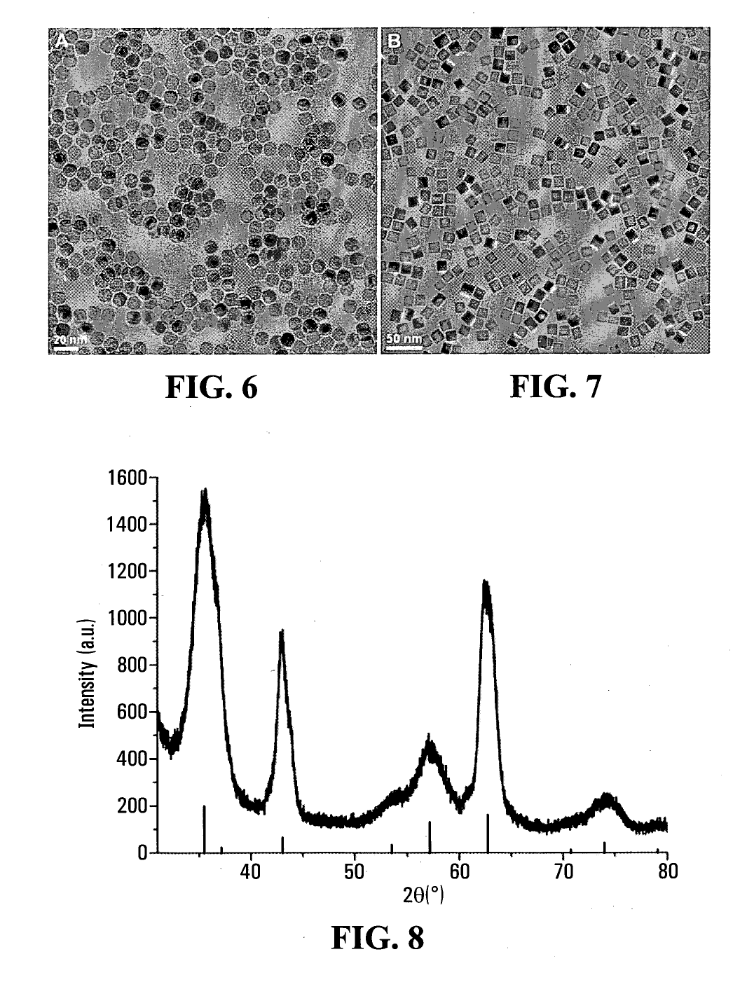

Representative TEM images of sample magnetite nanoparticles are shown in FIGS. 6 and 7 respectively.

XRD patterns of the magnetite nanoparticles indicated the presence of magnetite crystals. A representative XRD diffraction spectrum of sample magnetite particles is shown in FIG. 8. The peaks shown in FIG. 8 are consistent with inverse spinel magnetite phase, not hematite or wustite structures.

### Example II

### Preparation of Heterodimer Particles

Heterodimer nanoparticles were formed by reducing Ag in the presence of the sample magnetite nanoparticles prepared in Example I as seeds.

In a typical procedure, magnetite nanoparticles as prepared in Example I (40 mg), silver acetate (40 mg), oleylamine (0.5 ml), and 1,2-dodecanediol (0.1 g) were mixed in 20 ml of hexane or toluene. The reaction mixture was heated to about 60 to about 110° C. for about 10 to about 60 minutes under magnetic stirring. The initially black solution turned yellowish brown after reaction. Upon cooling to room temperature, ethanol (10 ml) was added to the reaction mixture to precipitate the heterodimer nanoparticles. A dark yellowish brown solid was collected by centrifugation at 6000 rpm, washed twice with hexane and ethanol, and magnetically decanted. The yellow supernatant solution containing mostly free Ag nanoparticles was discarded. The final product was redispersed in hexane or toluene for storage and further use.

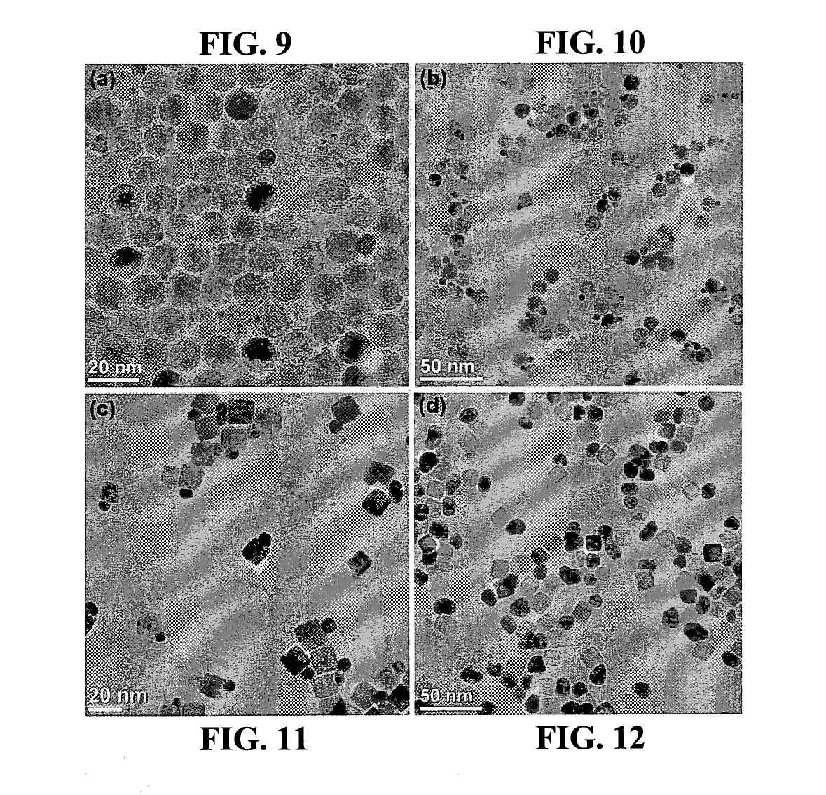

TEM images of representative sample heterodimer nanoparticles are shown in FIGS. 9, 10, 11, and 12. In these figures, the Ag component particles are shown as darker regions.

The reaction proceeded under mild conditions, and Ag particle (crystal) formation was visible at 60° C. within a few minutes. The sizes of the Ag component particles were tuned between about 2 and about 15 nm by changing reaction conditions, such as solvent type, temperature, and reducing reagents. Representative data showing the dependence of particle size on these conditions are listed in Table I.

TABLE I
Ag Particle Size Dependence on Reaction Conditions

| | | Ag | | Oleyl- | Oleic | | | |
|---|---|---|---|---|---|---|---|---|
| Size | Fe₃O₄ | acetate | Diol | amine | acid | Organic | Temp | Time |
| (nm) | (mg) | (mg) | (g) | (ml) | (ml) | Solvent | (° C.) | (min) |
| 1-2 | 40 | 20 | — | 0.2 | 0.2 | 20 ml | 60 | 30 |
| | | | | | | Hexane | | |
| 6-7 | 40 | 40 | 0.2 | 0.5 | 0.2 | 20 ml | 100 | 30 |
| | | | | | | Toluene | | |
| 12-14 | 40 | 60 | 0.2 | 0.5 | — | 20 ml | 60 + | 30 + |
| | | | | | | Toluene | 100 | 30 |

It was found that Ag particle size also depended on the molar ratio of seed magnetite particles to the Ag precursor/source. When a more strongly polar solvent (such as toluene) was used, Ag crystal growth was faster and larger Ag particles could be formed, as compared to a less strongly polar solver (such as hexane). The addition of oleic acid was found to hinder the growth rate of Ag crystal.

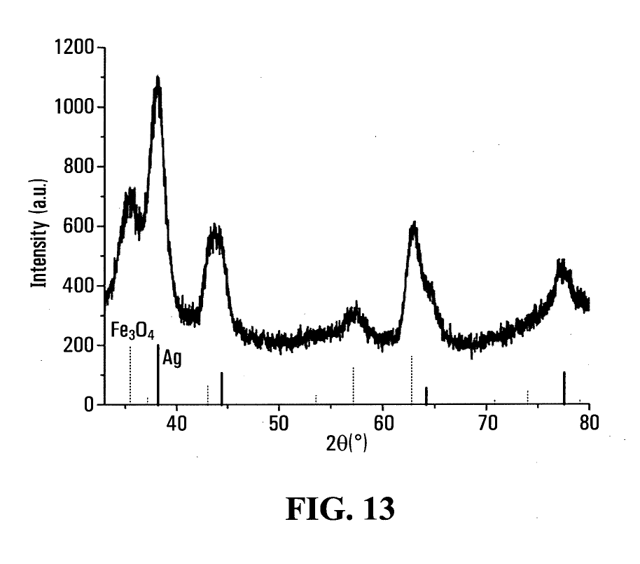

X-ray diffraction (XRD) patterns of the sheterodimer nanoparticles indicated the presence of magnetite and Ag crystalline phases. A representative XRD diffraction spectrum is shown in FIG. 13.

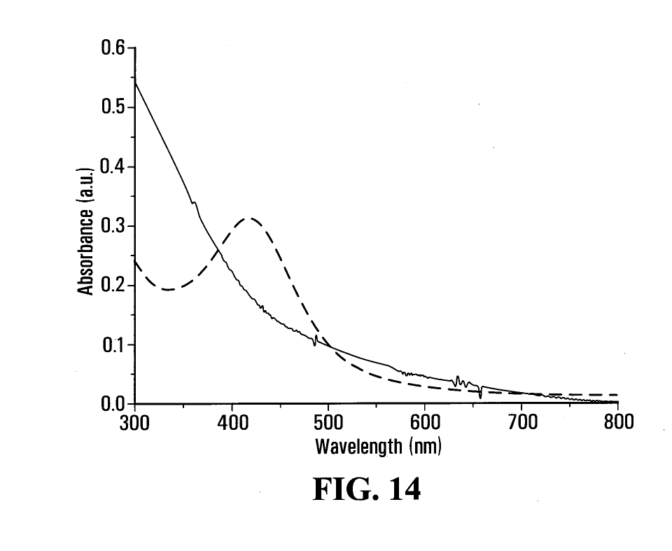

FIG. 14 is a representative UV-Vis absorption spectra taken from sample solutions of magnetite nanoparticles (solid line) and magnetite-Ag heterodimer nanoparticles (dashed-line) respectively. The absorption peak at 420 nm corresponded to surface plasmon absorption frequency of silver nanoparticles.

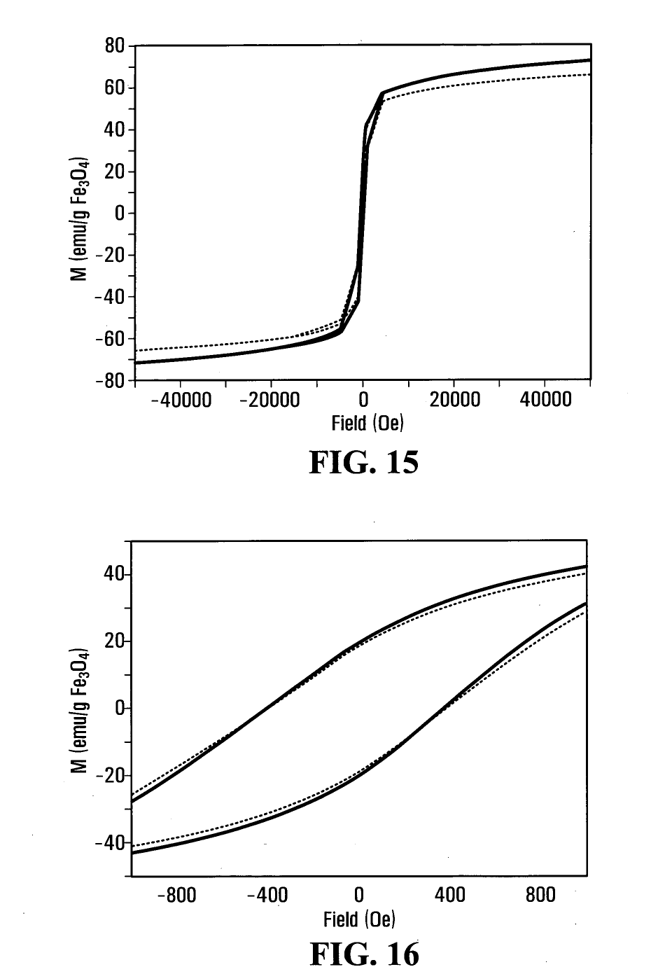

FIG. 15 shows the magnetic hysteresis loops of sample magnetite particles (solid line) and heterodimer particles (dotted line) respectively, which were measured at 6 K. FIG. 16 shows a portion of the loops at an expanded scale for coercivity measurements. The magnetite (component) particles used for these figures were of a particle size of about 13 nm. The Ag component particles were of a particle size of about 7 nm.

The sample heterodimer nanoparticles were found to be superparamagnetic with a blocking temperature of 225 K, and to have a magnetization (M) value of 66 emu/g Fe₃O₄ at an applied field (H) of 50 kOe at 6 K. The hysteresis loops for sample magnetite and heterodimer particles were similar, indicating the same coercivity (H_{c}) of 400 Oe. The presence of Ag appeared to have no significant effect on the magnetic properties of the magnetite component. Furthermore, the zero field cooled (ZFC) and field cooled (FC) curves measured under an applied field of 50 Oe showed no significant difference for the two types of sample particles.

### Example III

### Comparison Sample

For comparison purposes, comparison Ag nanoparticles were prepared as in Example II but without adding the seed magnetite nanoparticles.

Under similar reaction conditions, Ag reduction did not appear to occur until after at least 30 minutes of reaction time. This result indicates that the presence of the seed magnetite nanoparticles prepared in Example I can catalyze Ag reduction on their surfaces, thus significantly reducing production time.

### Example IV

### Surface Modification of Sample Particles

Sample heterodimer nanoparticles prepared in Example II were initially capped with a hydrophobic layer composed of oleic acid and oleylamine. These sample particles were soluble in organic non-polar solvents, such as hexane, toluene or chloroform.

The particle surfaces were modified with a ligand exchange procedure according to the scheme illustrated in FIG. 5. In this approach, the strong bindings between thiol groups and an Ag surface and between TMAH and an oxide surface were utilized. GSH was employed as the water-soluble thiol molecule for Ag surface passivation.

In a typical procedure, ethanol was added to a hexane dispersion of heterodimer nanoparticles, and the solution was magnetically decanted to produce a precipitate. An aqueous solution of 50 mM of GSH and 0.2 M of TMAH was added to this precipitate. The resulting mixture was shaken for 5 min. 2-propanol was then added to the mixture solution and the solution was magnetically decanted. After washing with acetone once, the product was redispersed in deionized water, forming a clear solution that was found to be stable over a period of several (e.g. up to six) months.

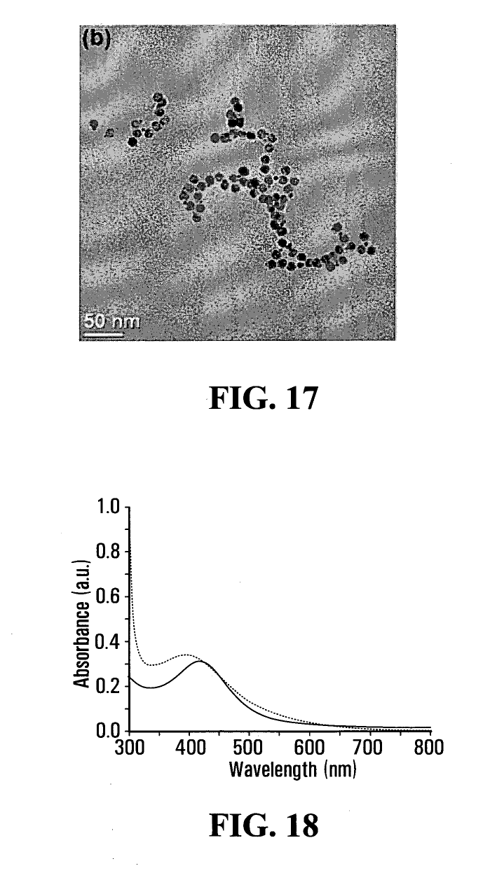

FIG. 17 is a representative TEM image of sample heterodimer nanoparticles after the ligand exchange. The sample nanoparticles remained highly dispersed, and the heterodimer structure was retained even after having been kept in buffer solutions for several days. FIG. 18 shows the ultra violet-visible (UV-Vis) absorption spectra for the sample heterodimer nanoparticles before (solid line) and after (dash line) the ligand exchange. It was found that the surface plasmon absorption peak of Ag was blue-shifted slightly due to the change in the local dielectric environment (i.e. the solvent was changed from hexane to water).

### Example V

### Cell Culture and Nanoparticle Uptake

Mouse macrophage cells (RAW274.6, ATCC) were cultured in RPMI-1640 culture medium, supplemented with fetal bovine serum (FBS) (10%), penicillin (200 units/ml) and streptomycin (200 μg/ml), and maintained at 37° C. in a humidified atmosphere containing 5% of CO₂. At confluence, the cells were washed, trypsinized and re-suspended in a culture medium. Cells were seeded at a concentration of 2×10⁴ cells/well on 12 mm-diameter glass coverslips in a 24-well tissue culture plate, and allowed to grow for 24 hours at 37° C. under 5% of CO₂. Sample nanoparticles as prepared in Example IV were loaded at different concentrations (1 to 10 μg/ml) into the culture medium. The cells were grown for a further 24 hours at 37° C. under 5% of CO₂.in the presence of the nanoparticles. Prior to imaging, the cells on the coverslips were washed with a fresh RPMI-1640 growth medium to remove free nanoparticles that were not attached to the cells.

### Example VI

### TPF Imaging and Magnetic Manipulation

After uptake of the heterodimer nanoparticles by macrophage cells, the cells were imaged using a TPF system similar to system 100 shown in FIG. 1 Strong two-photon fluorescence was observed upon excitation by femto-second infrared laser pulses of 900 nm.

For comparison, the cells were also imaged with TPF before loading the heterodimer nanoparticles. No significant autofluorescence was observed under similar experimental conditions or under even a higher excitation laser power. Likely, any autofluorescence was too weak to be seen under these experimental conditions.

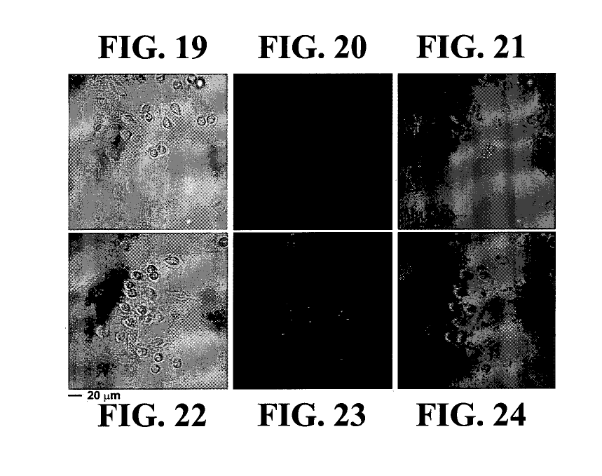

FIGS. 19 to 24 show representative images of the cells without (FIGS. 19, 20 and 21) or with (FIGS. 22, 23, and 24) loaded heterodimer nanoparticles. FIGS. 19 and 22 are bright-field images showing the cell outlines. FIGS. 20 and 23 are TPF images, which show that when the cells were labeled with sample nanoparticles they were detectable by TPF, and when the cells were not labeled with sample nanoparticles they were not detectable by TPF. FIGS. 21 and 24 are superimposed images of FIGS. 19 and 20, and 22 and 23, respectively. For these images, the cells were loaded with magnetite-Ag heterodimer nanoparticles in a culture medium containing 2 μg/ml of the heterodimer nanoparticles. The particle size was about 11 to about 13 nm.

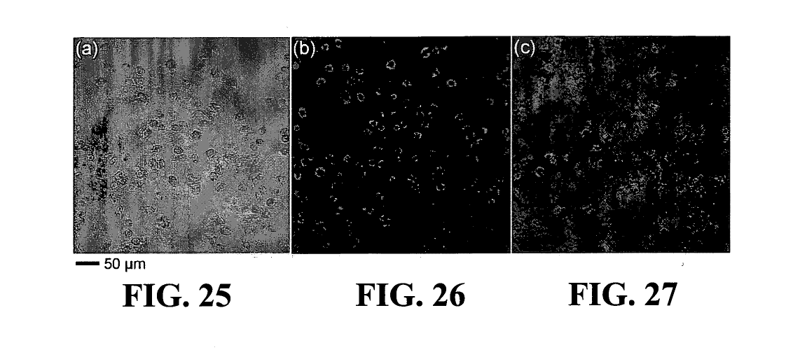

FIGS. 25, 26 and 27 show similar bright-field, TPF and superimposed images of cells loaded with magnetite-Ag heterodimer nanoparticles, at different magnification.

The sample heterodimer particles with Ag domains as small as about 10 to about 15 nm were found to be useful for TPF detection and imaging, such as imaging of target cells. It can be expected that these particles would also be useful for imaging other types of cells.

The cells labeled with sample heterodimer nanoparticles were also manipulated using a magnetic field. The large magnetic moment of the iron oxide domain in the heterodimer nanoparticles was conveniently utilized in this respect. For example, cellular rotation and translation in the presence of an NdFeB permanent magnet was effected and observed.

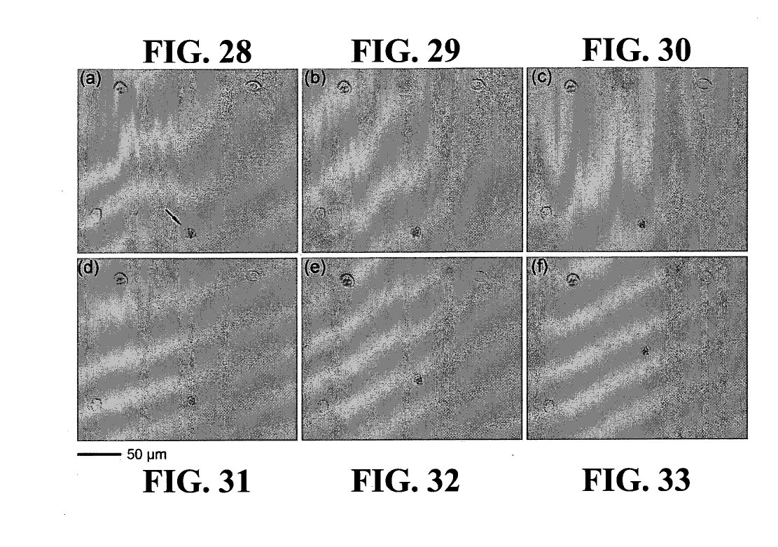

Manipulation of macrophage cells labeled with sample heterodimer particles using a local magnetic field is illustrated in FIGS. 28 to 33, which show representative sequential bright-field images of the cells at different times over a 10-second period. The subject cell is indicated by the arrow in FIG. 28, which is loosely attached to the supporting surface. FIG. 29 shows the same cell after it had been rotated from the position in FIG. 28. FIGS. 30 to 33 show the translation movement of the cell, which is evident from its position relative to the other fixed cells.

Other features, benefits and advantages of the embodiments described herein not expressly mentioned above can be understood from this description and the drawings by those skilled in the art.

Of course, the above described embodiments are intended to be illustrative only and in no way limiting. The described embodiments are susceptible to many modifications of form, arrangement of parts, details and order of operation. The invention, rather, is intended to encompass all such modification within its scope, as defined by the claims.

1. A method comprising:
irradiating a heterodimer particle with light of a wavelength selected to induce two-photon fluorescence emission in said particle, said heterodimer particle comprising a first component particle comprising magnetite, a second component particle comprising silver and having a structure and a particle size selected to generate said two-photon fluorescence emission in response to said irradiating; and
detecting said two-photon fluorescence emission from said heterodimer particle.

2. (canceled)

3. The method of claim 1, wherein said particle size of said second component particle is about 10 to about 15 nm.

4. The method of claim 1, said second component particle comprises a silver crystal.

5. The method of claim 1, wherein said second component particle is generally spherical.

6. The method of claim 1, wherein said heterodimer particle is attached to a target during said irradiating and said detecting.

7. The method of claim 1, wherein said heterodimer particle is inside a target during said irradiating and said detecting.

8. The method of claim 6, comprising imaging said target based on said detecting.

9. The method of claim 6, wherein said target is a cell.

10. The method of claim 1, wherein said heterodimer particle comprises tetramethylammonium hydroxide bonded to a surface of said first component particle, and comprises glutathione bonded to a surface of said second component particle.

11. The method claim 1, wherein said wavelength is about 800 to about 950 nm.

12. The method of claim 1, comprising moving said heterodimer particle with a magnetic force.

13. A particle comprising:
a first component comprising magnetite;
a second component comprising silver, said first and second components forming a heterodimer nanoparticle;
tetramethylammonium hydroxide bonded to a surface of said first component; and
glutathione bonded to a surface of said second component.

14. The particle of claim 13, wherein said second component is generally spherical.

15. The particle of claim 13, wherein said second component has a size of about 2 to about 15 nm.

16. The particle of claim 13, wherein said second component has a size of about 10 to about 15 nm.

17. The particle of claim 13, wherein said first component has a generally spherical or cubic shape.

18. A method comprising:
preparing a magnetite particle,
growing a silver particle on said magnetite particle, in the presence of a reducing agent, to form a heterodimer nanoparticle comprising said magnetite particle as a first component and said silver particle as a second component, wherein said reducing agent is 1,2-dodecanediol.

19. The method of claim 18, wherein said preparing said magnetite particle comprises heating iron oleate dissolved in 1-octadecene to a temperature of about 320° C.

20. The method of claim 19, wherein said iron oleate dissolved in said 1-octadecene is heated for about 30 minutes.

21. The method of claim 19, wherein said heating comprises heating said iron oleate dissolved in said 1-octadecene in the presence of oleic acid or sodium oleate.

22. The method of claim 18, wherein said growing comprises heating a precursor solution to a temperature from about 60 to about 110° C. for about 10 to about 60 minutes, said precursor solution comprising magnetite particles, a silver source, said 1,2-dodecanediol, and an organic solvent.

23. The method of claim 22, wherein said precursor solution comprises oleylamine.

24. The method of claim 22, wherein said organic solvent is hexane or toluene.

25. The method of claim 22, wherein said silver source is silver acetate.

26. A method of preparing a water-soluble particle from a heterodimer particle comprising a magnetite component and a silver component, comprising:
bonding tetramethylammonium hydroxide (TMAH) to a surface of said magnetite component; and
bonding glutathione (GSH) to a surface of said silver component.

27. The method of claim 26, comprising mixing said heterodimer particle, GSH, and TMAH in a solution.

28. The method of claim 27, wherein said solution is aqueous.
